(19) United States
(12) Patent Application Publication
(10) Pub. No.: US 2022/0161013 A1
(43) Pub. Date: May 26, 2022

(54) IMPROVED DELIVERY OF DRUG THERAPY TO THE CNS BY ULTRASOUND-BASED OPENING OF THE BLOOD-BRAIN BARRIER

(71) Applicant: NORTHWESTERN UNIVERSITY, EVANSTON, IL (US)

(72) Inventors: Daniel Y. Zhang, Lisle, IL (US); Roger Stupp, Chicago, IL (US); Adam M. Sonabend Worthalter, Chicago, IL (US)

(21) Appl. No.: 17/599,313

(22) PCT Filed: Apr. 3, 2020
(86) PCT No.: PCT/US2020/026614

§ 371 Date: Sep. 28, 2021

Related U.S. Application Data
(60) Provisional application No. 62/829,359, filed on Apr. 4, 2019.

(51) Int. Cl. A61M 37/00; A61K 31/337; A61K 9/00

(52) U.S. Cl. CPC A61M 37/0092; A61K 31/337; A61M 2205/04; A61K 9/0085; A61M 2210/0693; A61K 9/0009

(57) ABSTRACT

A method for treating central nervous system tumors is a patient comprising: treating the patient intravenously with therapeutic agent including Cremophor EL-free paclitaxel, and disrupting a blood-brain barrier within the brain of the patient by the use of an ultrasound device and intravenous microbubble injection. In other methods, the subject is treated with a therapeutic agent comprising an albumin-bound paclitaxel that crosses the blood brain barrier. In this manner, the disruption of the blood-brain barrier increases the concentration of paclitaxel in the brain as compared to the concentration of paclitaxel in the brain without blood-brain barrier disruption and intravenous microbubble injection.

### CROSS-REFERENCE TO RELATED APPLICATIONS

This application claims the benefit of U.S. Provisional Application No. 62/829,359, filed Apr. 4, 2019 which is incorporated herein by reference.

### STATEMENT AS TO FEDERALLY SPONSORED RESEARCH

This invention was made with government support under OD021356 awarded by NIH; under CA060553 awarded by NIH; and CA221747 awarded by NIH. The government has certain rights in the invention.

### BACKGROUND

Glioblastoma (GBM) remains an incurable disease and yet the most common and lethal type of brain tumor. As numerous novel therapeutic approaches have failed in controlled clinical trials, temozolomide (TMZ) remains the first-line drug in the treatment of these tumors, which is in part explained by its good penetration across the blood-brain barrier (BBB). Drug therapy in glioblastoma (GBM) frequently fail in patients despite showing good efficacy in preclinical models and in other cancers due to poor drug penetration across the blood-brain barrier (BBB).

Paclitaxel (PTX), a microtubule-stabilizing drug, is exquisitely potent against GBM in preclinical models. A previous study on three patients that had received preoperative PTX infusion showed only a minimal PTX concentration in the glioma tissue, it was undetectable in peri-tumoral brain tissue. The latter is an essential compartment for effective drug treatment of an infiltrative disease like glioma (Heimans et al). These results were in accordance with a distribution experiment by Lesser et al performed in rats using ³H-PTX, which failed to detect drug in the brain. Moreover, Fellner et al. showed that PTX is a substrate for the multidrug resistance protein p-Glycoprotein, an efflux pump highly expressed in brain capillary endothelial cells. When using a p-Glycoprotein blocker, valspodar, PTX showed significant increased effectiveness in orthotropic glioma xenograft model in nude mice. Collectively, these studies demonstrate that while PTX might partially penetrate into brain tumors due to deficient BBB, it is unable to cross the intact BBB in peri-tumoral brain tissue. Moreover, Cremophor EL, the solvent used in conventional PTX formulations is neurotoxic. Thus, whereas PTX remains one of the most potent drugs against GBM, it cannot be exploited due to poor BBB penetration and vehicle-related toxicity.

There exists a need for chemotherapeutic agents that are able to penetrate into the brain across the blood brain barrier and display effectiveness against glioblastoma cells.

### SUMMARY

In one aspect, a method for treating central nervous system tumors in a patient is provided. The method includes treating the patient intravenously with a Cremophor EL-free therapeutic agent, while disrupting a blood-brain barrier within the brain of the patient. BBB disruption may be achieved by the use of an ultrasound device and intravenous microbubble injection. In this manner, the disruption of the blood-brain barrier increases the concentration of paclitaxel in the brain as compared to the concentration of paclitaxel in the brain without blood-brain barrier disruption and intravenous microbubble injection.

In another aspect, a method for delivering a therapeutic agent includes an albumin-bound paclitaxel across the blood brain barrier to deliver the therapeutic agent to the brain of a subject.

The methods are useful for both primary and secondary central nervous system tumors, such as glioblastoma and lower grade gliomas, or metastatic brain tumors (e.g. from breast and lung cancer, melanoma and other solid tumors).

Other methods, features and/or advantages are, or will become, apparent upon examination of the following figures and detailed description. It is intended that all such additional methods, features, and advantages be included within this description and be protected by the accompanying claims.

### BRIEF DESCRIPTIONS OF DRAWINGS

FIG. 1 is a comparison of drug potency across glioma cell lines and patient derived explant cultures.

FIG. 2A-D demonstrate superior therapeutic efficacy and brain penetration of paclitaxel following IV infusion of albumin bound paclitaxel compared to paclitaxel dissolved in Cremophor EL.

FIG. 3A-D demonstrate the effect of brain sonication on paclitaxel levels.

FIG. 4A-B demonstrates survivability in an albumin bound paclitaxel administered following sonication treated xenograft model.

FIG. 5A-B demonstrates toxicity of therapeutic agents comprising Cremphor EL in non-tumor bearing mice.

### DETAILED DESCRIPTION

### I. Definitions

GBM refers to glioblastoma, formerly named glioblastoma multiforme. The methods described herein are effective for treatment of GBM, glioblastoma, malignant glioma, diffuse and anaplastic astrocytoma, oligodendroglioma, or any other primary or secondary central nervous system malignancy.

US refers to ultrasound. Pulsed-US refers to repeated and intermittent use of the US to provide ultrasound-based blood brain barrier disruption. For example, pulsed-US may refer to ultrasonic waves applied in 20 ms bursts every second for a total duration of 120 seconds. This may be considered one course of pulsed-US treatment. US-based delivery system refers to a combination of the US device, and a source of chemotherapeutic (including cytotoxic, biological, or targeted) agent. Typically, US-based delivery system refers to refers to an implanted US in conjunction with administration of intravenous micro-bubbles.

PTX refers to paclitaxel. ABX refers to albumin bound paclitaxel and may be available under the tradename Abraxane. The complex of PTX with albumin may also be described as a particle or nanoparticle albumin bound paclitaxel, or nab-paclitaxel. Albumin-bound PTX is ABX, or Abraxane® (Celgene). ABX does not require Cremophor EL (CrEL) as a solvent.

CrEL is an abbreviation for the solvent Cremophor EL; CrEL-PTX is a combination of Paclitaxel and Cremophor EL. Cremophor EL (CrEL), a 50:50 mix of polyethylated castor oil and ethanol used to dissolve unbound paclitaxel that has been shown to be neurotoxic. A CrEL free paclitaxel composition is a composition of paclitaxel that is non-toxic because it is substantially free of CrEL. Substantially free of CrEL indicates that amounts of CrEL are so low that toxic side effects normally associated with CrEL are nonexistent. Of course, compositions containing trace, minor, or small amounts of CrEL are encompassed as CrEL free paclitaxel therapeutic agents as described herein. For example if a paclitaxel stock comprises CrEL as a solvent in a concentrate, upon formulation of a final composition, the amount of CrEL is so significantly reduced as to be considered CrEL free. Preferably the CrEL free paclitaxel therapeutic agent comprises less than 10%, 1%, 0.1%, 0.001, or 0% CrEL.

BBB refers to any blood brain barrier existing in the brain and central nervous system.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When “only A or B but not both” is intended, then the term “only A or B but not both” will be employed. Thus, use of the term “or” herein is the inclusive, and not the exclusive use. As used in the specification and the claims, the singular forms “a,” “an,” and “the” include the plural. Finally, where the term “about” is used in conjunction with a number, it is intended to include ±10% of the number. For example, “about 10” may mean from 9 to 11. The term wt % is meant to describe a comparison of the weight of one compound to the weight of the whole composition expressed as a percent. It can also be described as wt. %, or (w/w) %. A (w/w) % indicated the amount of a component in relation to the total composition of the traction fluid. Reference to a numeral 5 is equivalent to 5.0.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein “levels” refers to a measurable amount of a protein or chemical in a sample. The sample may be a blood, plasma, or tissue sample, for example. A level may be measured in any unit, for example ng/mL or ng/mg, micromolar, a percent of a total, or any other quantity.

As used herein “treat”, “treating” refers to administering to a subject a therapeutic agent under conditions permitting enhanced accumulation of the therapeutic agent in the brain of a subject. The therapeutic agent may be Cremphor EL free-paclitaxel or nanoparticle albumin bound paclitaxel or a combination thereof.

As used herein “therapeutically effective amount” refers to an amount of a therapeutic agent that relieves (to some extent, as judged by a skilled medical practitioner) one or more symptoms of a disease or condition in a subject. Additionally, by “therapeutically effective amount” of a composition is meant an amount that reduces a measurement of a primary or secondary central nervous system tumor. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

As used herein, the term “treatment” refers to all actions that alleviate, ameliorate or relieve the symptoms of a primary or secondary central nervous system tumor. As used herein, the term “treatment” means administering a therapeutic agent of the present invention to ameliorate or relieve the symptoms of a primary or secondary central nervous system tumor.

The pharmaceutical composition may further include suitable carriers, excipients and diluents conventionally used in the production of pharmaceutical composition. The pharmaceutical composition may be formulated in the form of oral preparations such as powders, granules, tablets, capsules, suspensions, emulsions, syrups, aerosols or the like, external preparations, suppositories, and sterilized injection solutions according to a conventional method. Specific examples of carriers, excipients and diluents that can be included in the composition may include, but are not limited to, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oil, and the like. In the case of formulation, diluents or excipients such as fillers, extenders, binders, wetting agents, disintegrants, or surfactants, can usually be used.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. The “subject” may also include a “patient” that is a subject identified as having a primary or secondary central nervous system tumor.

### II. Ultrasound

Any appropriate US device for disruption of a blood brain barrier within the brain of a patient is usable for improved delivery of a therapeutic agent to the peri-tumoral and macroscopically non-involved areas brain. The US device may be surgically implanted, or applied externally and/or transcranially. The US device may remain outside the cranial cavity. Any appropriate frequency, form or duration of ultrasonic treatment of the brain that provides transient blood-brain barrier disruption can be used in the methods disclosed herein.

In some cases, the use of US in humans requires ultrasound waves to bypass the thick human skull. For example, to overcome this, an US device that is implanted into skull, or cranial, window has been successfully tested in a Phase 1 clinical trial for recurrent GBM with the chemotherapeutic agent carboplatin. In this way, the ultrasonic waves bypass the skull. The feasibility, safety, and acoustic pressure parameters of surgically implantable US devices have been established.

Once an implanted US device is connected to the external generator, these devices can generate a reproducible low intensity ultrasound field on the targeted brain tissue without MRI guidance, or feedback mechanisms as opposed to other transcranial US BBB-disruption technologies. With these devices, sonication is simple and accessible allowing for repetitive treatments. In particular, the variability that occurs due to acoustic transmission through the skull bone with external focused US does not exist with the implantable US technology, which allows for a known pressure output of the device. The implantable US technology is therefore easily adapted to treat broad areas, greatly simplifying the brain sonication procedure for large-scale studies and therapeutic BBB disruption with concomitant chemotherapy infusions. There are several differences and advantages of implantable US technology over US devices that transmit through the skull including: diffuse BBB opening zone in the brain, avoiding need for MM guidance (gliomas are a diffuse disease); lower infrastructure/setup, cost and logistical challenges relative to MM-guided transcranial US; lower emission energy than trans-skull systems, since there is no skull energy attenuation; known acoustic pressure output in brain tissue; and ease of sonication allowing for repetitive drug therapy over many cycles.

### III. Therapeutic Agents

Albumin bound paclitaxel or CrEL-free paclitaxel is currently approved for use in metastatic breast cancer and non-small cell lung cancer and pancreatic cancer. Many cancers express high levels of SPARC and gp60, which are proteins that bind to albumin and may transfer albumin into the cell. Furthermore due to the fact Abraxane® is protein bound, it is dissolvable in water, which removes the need for CrEL. CrEL also has been demonstrated to reduce bioavailability of PTX by trapping PTX in the plasma compartment of the blood through the formation of micelles.

Local delivery of PTX through convection-enhanced delivery showed 73% response rate, but was associated with toxicity attributed to the delivery method (Lidar et al). Moreover, several studies showed that Cremophor EL (CrEL), the solvent used in conventional PTX formulations, accumulates in, and is toxic for, peripheral nerves, whereas PTX was not necessarily detectable in the tissue.

An FDA-approved formulation of albumin-bound PTX (ABX) or a CrEL free paclitaxel is well tolerated and exhibits better brain penetration than conventional PTX. Following systemic ABX administration into mice, US-based BBB disruption increased PTX brain tissue concentrations by 5-fold, achieving substantially higher levels than IC50 concentrations for most glioma cell lines.

### IV. Concomitant Treatment with Ultrasound and Biodistribution of a Therapeutic Agent

In one aspect described herein is a method for delivery of a therapeutic agent across a blood brain barrier in a subject including: treating the subject with a therapeutic agent comprising CrEL-free paclitaxel, while (either before or after) disrupting a blood-brain barrier within the brain of the subject by the use of an ultrasound device. By this method, the disruption of the blood-brain barrier increases the concentration of therapeutic agent in the brain of the subject as compared to the concentration of paclitaxel in the brain without blood-brain barrier disruption.

In some aspects, the step of treating the subject comprises administration of the therapeutic agent intravenously.

In some aspects, the therapeutic agent comprises nanoparticle albumin bound CrEL-free paclitaxel.

In some aspects, disruption of the blood-brain barrier comprises coincident application of an ultrasound device and intravenous microbubble injection.

In some aspects, the ultrasound device directs blood-brain barrier disruptions in a peri-tumoral region of the brain to increase the concentration of the therapeutic agent in the peri-tumoral region of the brain.

In some aspects, the ultrasound device applies ultrasonic waves transcranially.

In some aspects, the ultrasound device is implantable in a cranial window of the skull of the subject. The implantation of the ultrasound device is surgical.

In some aspects, the disruption of the blood-brain barrier comprising repeatedly activating, or pulsating the ultrasound device. In some aspects, the ultrasound device is applied externally.

In some aspects, the central nervous system tumor is glioblastoma, malignant glioma, diffuse or anaplastic astrocytoma, oligodendroglioma, or any other primary or secondary central nervous system malignancy. In some aspects, the subject having recurrent glioblastoma. In some aspects, the subject is suffering from primary or secondary brain tumors, and treatment beyond the blood brain barrier is indicated.

In some aspects, a method of treating primary or secondary central nervous system tumors in a subject includes treating the subject with a therapeutic agent. The therapeutic agent may be, but is not limited to, Cremophor EL-free paclitaxel. The method further includes disrupting a blood-brain barrier within the brain of the subject by the use of an ultrasound device. In this manner, the disruption of the blood-brain barrier increases the concentration of therapeutic agent in the brain as compared to the concentration of paclitaxel in the brain without blood-brain barrier disruption.

In some aspects, a method for delivery of a therapeutic agent across a blood brain barrier in a subject is provided. The method including treating the subject with a therapeutic agent including an albumin-bound paclitaxel, so that the therapeutic agent crosses the blood brain barrier to deliver the therapeutic agent.

In some aspects, the step of treating the subject includes administering the therapeutic agent intravenously. In some aspects, the therapeutic agent includes Cremophor EL free nanoparticle albumin bound paclitaxel. In some aspects treatment with the therapeutic agent may occur coincident application of an ultrasound device and intravenous microbubble injection. In some aspects, the subject is diagnosed with a central nervous system tumor is glioblastoma, malignant glioma, diffuse or anaplastic astrocytoma, or any other primary or secondary central nervous system malignancy. In some aspects, the subject has recurrent glioblastoma or is suffering from primary or secondary brain tumors, and treatment beyond the blood brain barrier is indicated.

In some aspects a method of treating primary or secondary central nervous system tumors in a subject is provided. The method including treating the subject with a therapeutic agent comprising an albumin-bound paclitaxel, wherein the therapeutic agent crosses the blood brain barrier to deliver the therapeutic agent to the primary or secondary central nervous system tumor.

Having demonstrated the efficacy of concomitant blood brain barrier disruption, intravenous microbubble injection, and intravenous nanoparticle bound paclitaxel administration, it is contemplated that paclitaxel or another chemotherapeutic or biological agent may be present in the tumor and surrounding tissue in a higher concentration than without BBB disruption, thus this treatment allows for better crossing of the therapeutic agent(s) in the brain and tumor. For example, a CrEL-free paclitaxel solution, paclitaxel bound to a fragment of albumin or another protein, paclitaxel may be present in any nanobody or liposome, nanoparticle or other carrier.

### EXAMPLES

Certain embodiments are described below in the form of examples. While the embodiments are described in considerable detail, it is not the intention to restrict or in any way limit the scope of the appended claims to such detail, or to any particular embodiment.

### Example 1: Albumin-PTX (ABX) Effectiveness

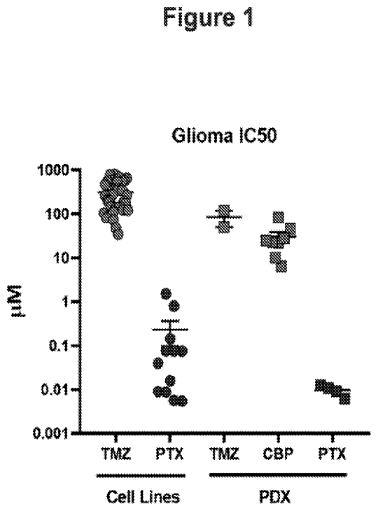

Referring now to FIG. 1, the effectiveness of PTX is demonstrated. In FIG. 1 a comparison of drug potency based on IC50 drug concentrations across glioma cell lines from Sanger/CCLE database (PTX and TMZ) and patient derived explant cultures from our lab (TMZ, PTX, and carboplatin) shows PTX is up to 1400-fold more potent than TMZ, and 120-fold more potent than carboplatin.

### Example 2: ABX Brain Penetration and Tolerance

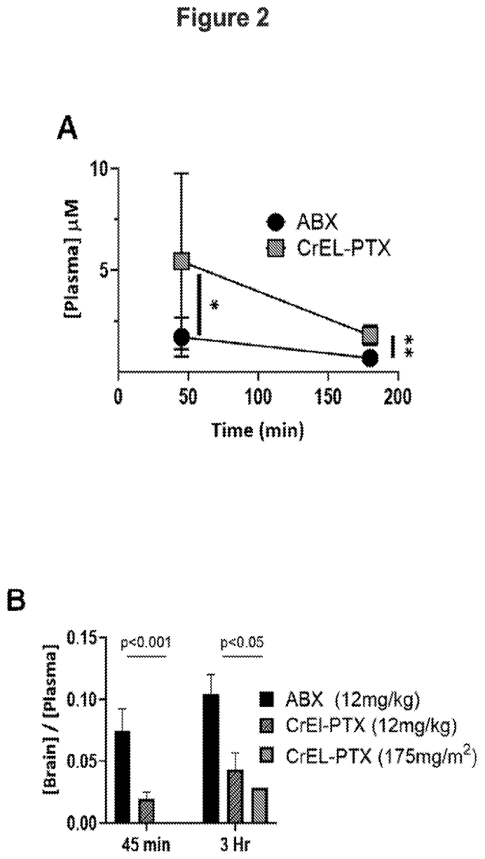

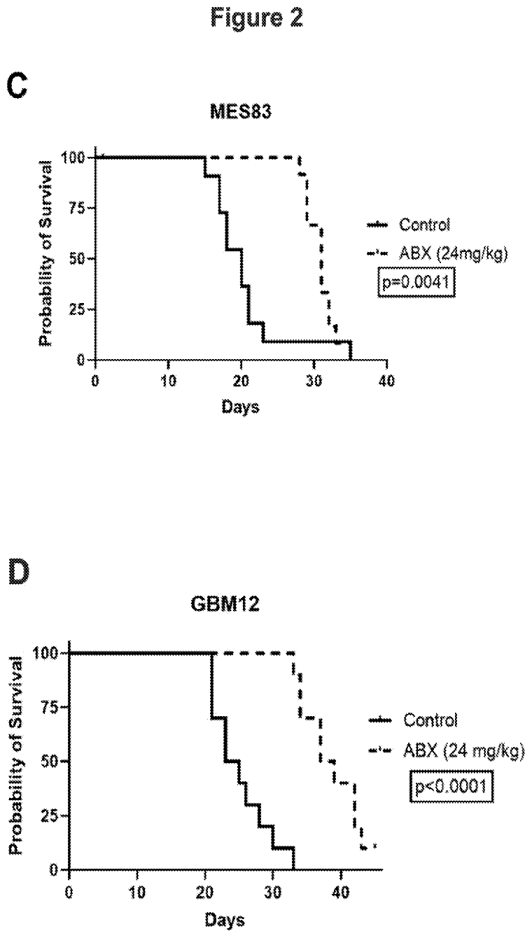

To minimize the chances of central nervous system (CNS) toxicity, we have compared delivery of CrEL-PTX and ABX. Following the same PTX dose of 12 mg/kg IV infusion in mice, ABX exhibited lower plasma concentrations compared to CrEL-PTX over time (FIG. 2A). Interestingly, brain/plasma concentration ratios were significantly higher for mice treated with ABX compared to mice treated with CrEL-PTX, or to the ratio seen in human peri-tumoral brain following preoperative CrEL-PTX 175 mg/m² infusion (FIG. 2B) reported by Heimans et al. Moreover, following US-based BBB disruption, ABX also led to higher brain concentrations than CrEL-PTX.

Without wishing to be bound by theory, this effect might be explained by SPARC and GP60, protein/amino acid transporters over-expressed by tumors and endothelium, which enhance BBB penetration and glioma cell uptake of albumin-bound drug-loaded nanoparticles. Consistent with this, we performed a survival analysis of patient-derived intracranial glioma xenografts (PDX) in mice treated with IV ABX, and found that this treatment was associated with a significant survival benefit in the absence of US delivery (FIG. 2C).

Referring now to FIG. 2A-D, FIG. 2A-B are a comparison of paclitaxel levels following IV infusion of paclitaxel with CrEL vehicle (CrEL-PTX) versus albumin-bound paclitaxel (ABX) in the absence of sonication for plasma (FIG. 2A), and ratio of brain/plasma levels (FIG. 2B) following IV infusion of 12 mg/kg in mice. In FIG. 2C, survival analysis of mice bearing intracranial patient-derived glioma xenografts treated with IV ABX 24 mg/kg on days 5, 7, 9, 11, 16, 18, 20 and 22 after tumor implantation shows this drug has efficacy in the absence of sonication in two independent experiments using two different patient-derived xenograft models (MES83 and GBM12). P value was determined using Log-rank test.

### Example 3: The Effect of Brain Sonication on PTX Levels

To investigate the effect of BBB disruption on PTX concentrations in the brain, mice without tumors were split into two groups. One group underwent ultrasound therapy in order to disrupt blood brain barrier. Immediately following ultrasound therapy, mice were injected with 12 mg/kg dose of ABX (Abraxane®) with a 20 mg/kg dose of fluorescein. A second group received the same injections without ultrasound therapy to serve as a control. Fluorescein was used as a fluorescent agent to map blood brain barrier disruption. After 45 minutes or 180 minutes mice were sacrificed and their brains were collected and viewed under a fluorescent microscope. Areas of the brain that displayed high areas of fluorescence were separated from non-fluorescent areas of the brain. Highly fluorescent areas of the brain were considered to be areas of the brain where blood brain barrier was disrupted or permeabilized. These samples were sent in for LCMS in order to determine drug concentration. It was determined that at 45 and 180 minutes, fluorescent regions of the brain showed 2-5 fold higher brain:plasma ratio compared to nonfluorescent and control brains. Furthermore the concentration of Abraxane® found in fluorescent regions of the brain was nearly 3 to 100 fold higher than the IC50 value for 10 out of 12 glioma cell lines.

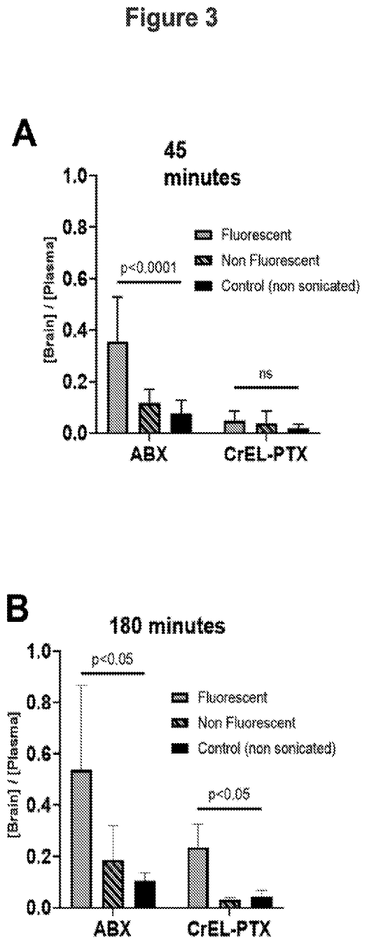

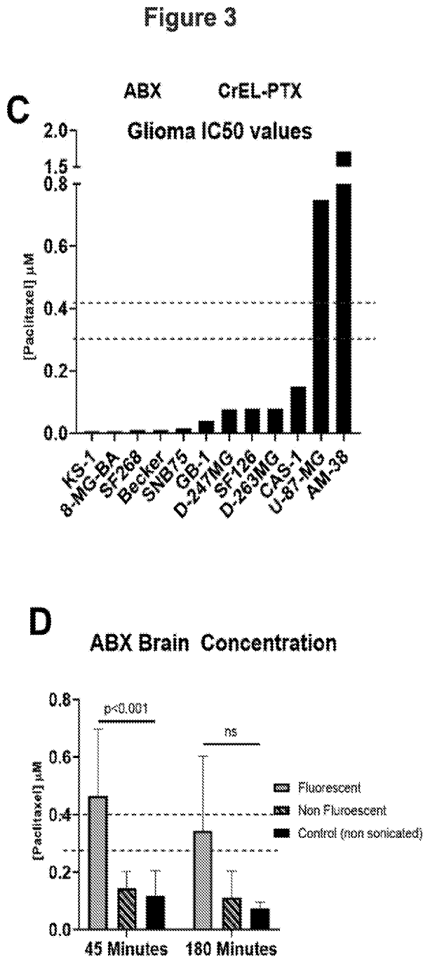

Referring now to FIG. 3, the effect of brain sonication on PTX levels in the brain was determined using fluorescein to map BBB disruption in mice injected with either ABX or CrEL-PTX 12 mg/kg IV, and compared to non-fluorescent brain regions and non-sonicated mouse brains as controls. Brain/plasma PTX concentration ratios were determined at 45 minutes (FIG. 3A) or 3 hrs (FIG. 3B). Susceptibility of human glioma cell lines to PTX illustrated by IC50 concentration from Sanger/CCLE database (n=12) (FIG. 3C) is shown in the context of PTX brain concentrations achieved following IV injection of 12 mg/kg of IV ABX (FIG. 3D). Significance was determined One-way ANOVA test.

### Example 4: The Effect of US Delivered ABX on Survival

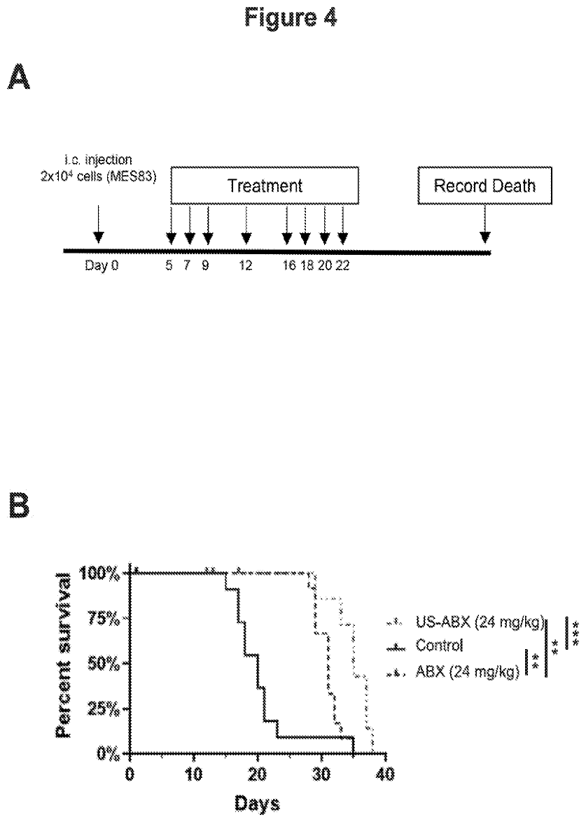

Referring now to FIG. 4, the MES83 xenograft model previously described was used to examine the effect US delivered ABX (Abraxane®) has on survival. Treatment began 5 days after implantation surgery and followed the same 3×/week schedule (MWF) for two weeks as previously described. US delivered ABX (Abraxane®) was able to extend median survival from 20 days to 35 days compared to control groups.

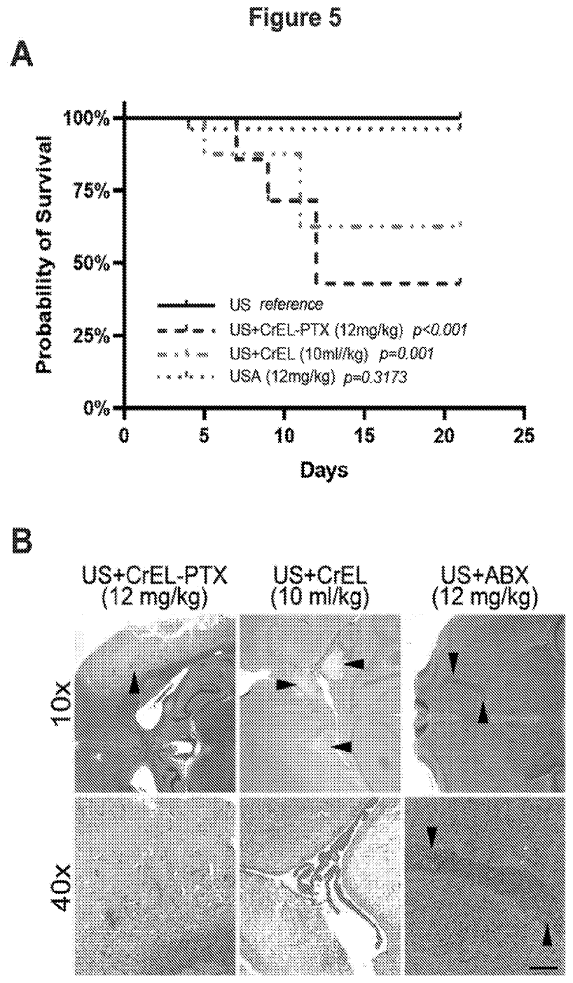

In some cases, given the lower rate of myelosuppression and neuropathy, ABX is usually given in doses up to 260 mg/m2 q 3-weeks with a lower incidence of myelosuppression and neuropathy than previously observed with PTX. Referring now to FIG. 5, non tumor bearing mice were subjected to multiple courses (up to 8) of treatment of US, US with PTX (CrEL-PTX, 12 mg/kg), US with CrEL (5% in saline), or US with ABX (Abraxane®, 12 mg/kg). Moreover, repeated administration of IV ABX (12 mg/kg) with concomitant US-based BBB disruption 3 times per week for 2 weeks was found to be well tolerated.

On the contrary, repeated administration of IV CrEL-PTX with concomitant US-based BBB disruption 3 times per week for 2 weeks was found to lead to a 57% mortality rate and neurotoxicity in the form of diffuse white matter damage. Furthermore, when mice were administered IV CrEL alone, without PTX, the observed mortality rate was 37.5% with similar signs of neurotoxicity. This data suggests that CrEL, the vehicle solvent used in traditional PTX formulations, is the main cause of the CNS toxicity seen in mice administered IV CrEL-PTX with concomitant US-based BBB disruption.

In some aspects, it is expected that human patients having or suspected of having a central nervous system tumor can be treated with a CrEL-free paclitaxel, or ABX, for example at a dose level of 175 mg/m2 (Dose 1). This dose administration may be concomitant with US administration or not. Doses of 215 mg/m2 (Dose 2) and 260 mg/m2 (Dose 3) may also be acceptable. From that point the dosage may be repeated, escalated or deescalated. Since the therapeutic agent administered to a human does not contain CrEL, toxic side effects should not be anticipated. Further the methods described herein result in a therapeutic that is able to cross a blood brain barrier and reach a peri-tumoral region of the subject's brain, thereby permitting effective treatment of a brain or other central nervous system tumor.

As stated above, while the present application has been illustrated by the description of embodiments, and while the embodiments have been described in considerable detail, it is not the intention to restrict or in any way limit the scope of the appended claims to such detail. Additional advantages and modifications will readily appear to those skilled in the art, having the benefit of this application. Therefore, the application, in its broader aspects, is not limited to the specific details and illustrative examples shown. Departures may be made from such details and examples without departing from the spirit or scope of the general inventive concept.

1. A method for delivery of a therapeutic agent across a blood brain barrier in a subject comprising:
treating the subject with a therapeutic agent comprising Cremophor EL free paclitaxel, and
disrupting a blood-brain barrier within the brain of the subject by the use of an ultrasound device,
wherein, the disruption of the blood-brain barrier increases the concentration of therapeutic agent in the brain of the subject as compared to the concentration of paclitaxel in the brain without blood-brain barrier disruption.

2. The method of claim 1, wherein the step of treating the subject comprises administration of the therapeutic agent intravenously.

3. The method of claim 2, wherein the step of disrupting a blood brain barrier occurs at a period of time after intravenous administration of the therapeutic agent.

4. The method of claim 1, wherein the therapeutic agent comprises Cremophor EL free nanoparticle albumin bound paclitaxel.

5. The method of claim 1, wherein disruption of the blood-brain barrier comprises coincident application of an ultrasound device and intravenous microbubble injection.

6. The method of claim 1, wherein the ultrasound device directs blood-brain barrier disruptions in a peri-tumoral region of the brain to increase the concentration of the therapeutic agent in the peri-tumoral region of the brain.

7. The method of claim 1, wherein the ultrasound device applies ultrasonic waves transcranially.

8. The method of claim 1, wherein the ultrasound device is implantable in a cranial window of the skull of the subject.

9. The method of claim 8, wherein the implantation of the ultrasound device is surgical and is coincident with tumor removal.

10. The method of claim 1, wherein the disruption of the blood-brain barrier comprising repeatedly activating, or pulsating the ultrasound device.

11. The method of claim 1, wherein the ultrasound device is applied externally.

12. The method of claim 1, wherein the subject is diagnosed with a central nervous system tumor is glioblastoma, malignant glioma, diffuse or anaplastic astrocytoma, or any other primary or secondary central nervous system malignancy.

13. The method of claim 12, the subject having recurrent glioblastoma.

14. The method of claim 1, wherein the subject is suffering from primary or secondary brain tumors, and treatment beyond the blood brain barrier is indicated.

15. A method of treating primary or secondary central nervous system tumors in a subject comprising:
treating the subject with a therapeutic agent comprising Cremophor EL free paclitaxel, and
disrupting a blood-brain barrier within the brain of the subject by the use of an ultrasound device,
wherein, the disruption of the blood-brain barrier increases the concentration of therapeutic agent in the brain as compared to the concentration of paclitaxel in the brain without blood-brain barrier disruption.

16.-18. (canceled)

19. The method of claim 15, wherein the step of treating the subject comprises administration of the therapeutic agent intravenously.

20. The method of claim 15, wherein the therapeutic agent comprises Cremophor EL free nanoparticle albumin bound paclitaxel.

21. The method of claim 15, wherein disruption of the blood-brain barrier comprises coincident application of an ultrasound device and intravenous microbubble injection

22. The method of claim 15 wherein the central nervous system tumor is glioblastoma, malignant glioma, diffuse or anaplastic astrocytoma, or any other primary or secondary central nervous system malignancy.

23. (canceled)

24. The method of claim 15, the subject having recurrent glioblastoma.

25.-29. (canceled)
